(19) United States
(12) Patent Application Publication
(10) Pub. No.: US 2009/0275116 A1
(43) Pub. Date: Nov. 5, 2009

(54) Metallic PCR frames

(76) Inventor: Venugopal Subramanyam, Fremont, CA (US)

Correspondence Address: Venugopal Subramanyam, 3399 Warren Avenue, Fremont, CA, 94538, US

(21) Appl. No.: 12/150,552

(22) Filed: Apr. 30, 2008

(51) Int. Cl. C12M 1/34

(52) U.S. Cl. 435/287.2

(57) ABSTRACT

The present invention is represented by a two part PCR well plate. Part one, the outer shell, is constructed from a thin metal.. Part two, the tubes, are thermoformed in an array of 96/384 or formed individually and inserted or welded into each tube cell location. The use of a rigid metal frame for the PCR array virtually eliminates all warping problems associated with temperature cycling.

This invention along with certain methods of manufacture significantly contributes to the deterministic consistency of PCR results and the overall cycle time. The ability to achieve a significant reduction in costs is realized from the purchase of reusable PCR frames as well as the savings obtained from the costs associated with a reduction in disposal of biologically contaminated materials.

### FIELD OF THE INVENTION

The present invention relates generally to the disposable tube arrays utilized in the amplification of DNA fragments by a process referred to as PCR (polymerase chain reaction) and applications of the same.

### BACKGROUND OF THE INVENTION

The polymerase chain reaction (PCR) is a technique widely used in molecular biology. It derives its name from one of its key components, a DNA polymerase used to amplify a piece of DNA by in vitro enzymatic replication. As PCR progresses, the DNA thus generated is itself used as template for replication. This sets in motion a chain reaction in which the DNA template is exponentially amplified. With PCR it is possible to amplify a single or few copies of a piece of DNA across several orders of magnitude, generating millions or more copies of the DNA piece.

Almost all PCR applications employ a heat-stable DNA polymerase. The DNA polymerase enzymatically assembles a new DNA strand from DNA building blocks, the nucleotides, using single-stranded DNA as a template and DNA oligonucleotides required for initiation of DNA synthesis. The vast majority of PCR methods use thermal cycling, i.e., alternately heating and cooling the PCR sample to a defined series of temperature steps. These thermal cycling steps are necessary to physically separate the strands (at very high temperatures) in a DNA double helix (DNA melting) used as template during DNA synthesis (at lower temperatures) by the DNA polymerase to selectively amplify the target DNA. The power and selectivity of PCR are primarily due to selecting primers that are highly complementary to the DNA region targeted for amplification, and to the thermal cycling conditions used.

Developed in 1983 by Kary Mullis, PCR is now a common and often indispensable technique used in medical and biological research labs for a variety of applications.

The PCR process is typically run in an array of 96 or 384 well tubes that are molded into place in a suitable well frame. During the DNA amplification process the trays are placed into a thermal transfer device and cycled 40 to 60 times through several temperature stages, some exceeding 200 degrees Fahrenheit. Currently, the well tubes and frame are manufactured from injected molded plastic. During the heating and cooling cycles the plastic well plates warp. This uneven concave or in some cases convex warping of the well plates is causative to downstream processing problems particularly associated with automated dispensing equipment. Thin plastic pipette tips are moved into and out of the PCR tubes to dispense, suction and mix the chemistry within. The tips are automatically inserted into the PCR tubes to a distance as close to the tube bottom as the bottom diameter of the tip will allow in order to recover as much of the solution as possible. If the PCR microplate is warped the pipette tips are damaged as they are robotically moved to the bottom of the tubes. To date, attempts to deal with the warping problem have been made with PCR plates constructed of a thicker, harder plastic outer shell platform with a separate molding of the tube. Although this method has improved the warping issue, it has not eliminated the problem.

Successful PCR processing relies on precision temperature (±1° C.) control that is consistent from well tube to tube. Injection molded well plate arrays of 96 or 384 tube locations have inherent problems with consistent wall thickness from one tube location to another. This is due to the problems associated with the physics of plastic injection molding. Additionally, the minimum wall thickness of the reaction tubes achievable with plastic injection molding is at best 0.3 mm. It is therefore obvious that thinner and more consistent tube wall thicknesses can achieve a faster and more accurate temperature delta from setting to setting and improve consistency of reaction from tube to tube within the array.

Under certain processing conditions problems with electrical charge build up of the plastic well tube plate can occur. All currently utilized plastic materials used for PCR well plate manufacture are non-conductive plastics having poor ESD (electrostatic discharge) capability.

Contamination of the well tubes is of considerable concern in achieving accurate and consistently reliable results. In most cases the PCR well plates are disposed of after one use. This can be very expensive, not only due to the cost of the plastic PCR microplates but also the cost associated with the disposal of biological waste materials.

Therefore, a heretofore unaddressed need exists in the art to address the aforementioned deficiencies and inadequacies

### BRIEF SUMMARY OF THE INVENTION

The present invention is represented by a two part PCR well plate. Part one, the outer shell, is constructed from a thin metal, such as stainless steel, aluminum or copper. Part two, the tubes, are thermoformed in an array of 96/384 or formed individually and inserted into each tube well location.

The outer metal shell construction is designed for maximum rigidity by the use of a method for perforating the 96 (FIG. 1) or 384 (FIG. 3) well holes such that they consist of an outer collar (FIG. 5) located at the peripheral top or bottom surface of the hole. The collar is so constructed that it maintains a flat parallel surface to the overall plate and provides a registration and alignment surface as well as a locking mechanism for each tube inserted therein.

The metal outer frame is designed such that either the individual plastic tubes or the 96/384 tube array mat (FIGS. 2,4) can be inserted and held in place by a dimensional interference between the outside diameter of the tube and the inside diameter of the frame hole collar. This allows for the tubes to be pushed out of the frame and a new set of tubes inserted by means of a tool specifically designed for such a purpose. The ability to achieve a significant reduction in costs is realized from the purchase of reusable PCR frames as well as the savings obtained from the costs associated with a reduction in the disposal of biologically contaminated materials.

This invention along with certain methods of manufacture significantly contributes to the deterministic consistency of PCR results and the overall cycle time. This improvement is realized by an ability to maintain consistency of thermal transfer throughout the entire 96 or 384 well array. This is accomplished by the use of individual or arrayed tubes that are thermoformed with considerably thinner and more consistent wall thicknesses along with an efficient thermally conductive tube frame. The improved thermal conductance of the metal frame maintains temperature uniformity throughout the tube array and assists in speeding up the cycle time to achieve each temperature hold point at which the specific enzyme is active.

The use of a rigid metal frame for the PCR array virtually eliminates all warping problems associated with temperature cycling. This is extremely important to robotic dispensing steps used in other downstream processing. Warped PCR array plates are causative to a bottoming out and damaging of the dispenser tips used in subsequent robotic operations. Unnoticed damaged dispenser tips can adversely affect process results.

The invention of a rigid metal PCR frame also provides for electrical grounding, thus eliminating any electrostatic charge build up from occurring during the DNA amplification process. Large stray electrostatic charges can have an adverse affect on process results.

These and other aspects of the present invention will become apparent from the following description of the preferred embodiments taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 Metal fabricated 96 Well PCR microplate

FIG. 2 96 unit array of PCR well plastic tubes

FIG. 3 Metal fabricated 384 Well PCR microplate

FIG. 4 384 unit array of PCR well plastic tubes

FIG. 5 Top surface of Metal Plate: shows perforated collar

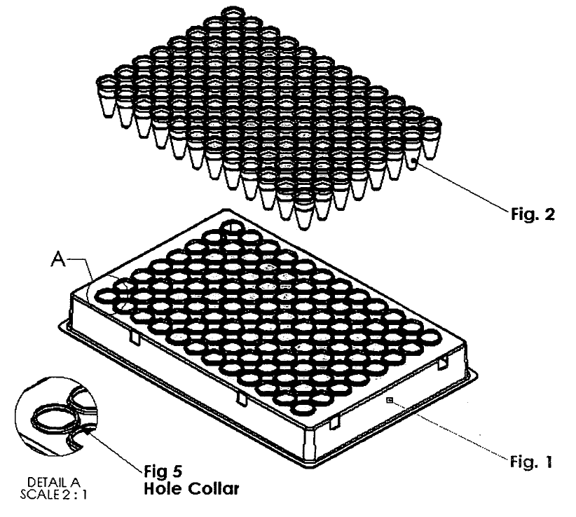

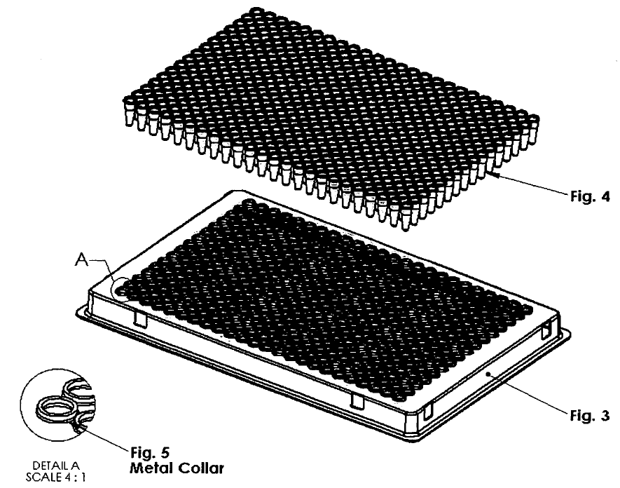

1. The utilization of a metal frame to hold individual PCR tubes (96 or 384)
The metal frame will significantly improve;
1.0 Thermal management: improved thermal cycling time and more consistent tube to tube temperature variation.
2.0 Reduction in plate warping. The metal frame produced by extruding an upward or downward drawn collar at each tube hole location provides for a stiff and flat surface to locate each tube. Warping of plastic PCR micro-plates is causative to significant down stream dispensing problems with automatic equipment.
3.0 Grounding for ESD (electrostatic discharge). The use of a metal frame significantly improves grounding of any electrical charge that may build up during the PCR process.
4.0 Significant reduction in plastic waste (spent tubes vs spent frames & tubes). The metal frames are re-usable.

2. The use of disposeable, individually or arrayed, plastic tubes.
Individual or arrayed PCR tubes that are press fitted into the metal frame collars, thus allowing the tubes to be mechanically discharged from the frame after use. This provides for a significant reduction in plastic waste and a significant cost improvement to the customer as the metal frames can be reused.

3. Consistent thin walled PCR tubes
Thermoformed thin walled plastic tubes insure a faster and more reliable temperature cycling of the PCR reaction. Temperature overshoot is greatly reduced. The use of disposable thin walled tubes or tube arrays is not possible without the use of a rigid metal frame as described in claim 1. Thin tubes or tube arrays without the use of a rigid and thermally stable frame will warp.
